(19) United States
(12) Patent Application Publication
(10) Pub. No.: US 2020/0108126 A1
(43) Pub. Date: Apr. 9, 2020

(54) FORMULATION AND METHOD FOR PROMOTING HAIR GROWTH

(71) Applicant: SKIN ACTIVES SCIENTIFIC LLC, Gilbert, AZ (US)

(72) Inventor: Hannah Naomi Sivak, Gilbert, AZ (US)

(21) Appl. No.: 16/152,742

(22) Filed: Oct. 5, 2018

(51) Int. Cl. A61K 38/44; A61K 38/18; A61K 9/00; A61P 17/14

(52) U.S. Cl. CPC A61K 38/44; A61P 17/14; A61K 9/0014; A61K 38/18

(57) ABSTRACT

The present application provides formulations and methods for promoting hair growth, including on the scalp, eyebrows, eyelashes and face. In embodiments, the formulations contain a combination of keratinocyte growth factor and methionine sulfoxide reductase.

### FIELD OF THE INVENTION

The present application provides formulations and methods for promoting hair growth, including on the scalp, eyebrows, eyelashes and face. In embodiments, the formulations contain a combination of keratinocyte growth factor and methionine sulfoxide reductase.

### BACKGROUND OF THE INVENTION

Hair loss in both male and female patients continues to be a significant problem for both young and ageing populations. While a number of compositions have been developed throughout the years, many pose problems to one or both sexes, including lotions and sprays containing finasteride, which can lead to serious side effects including, for men, increased risk for impotence, erectile dysfunction, decreased libido, and ejaculation disorder for the first year of treatment.

What is needed is a formulation that addresses the need to promote hair growth not only on the scalp, but also the eyebrows, eyelashes and face, while limiting unwanted side effects.

### BRIEF SUMMARY OF THE INVENTION

The present invention fulfills these needs by providing a topical formulation for promoting hair growth. Suitably, the topical formulation includes about 0.00002% (w/w) to about 0.00010% (w/w) of keratinocyte growth factor, about 0.00002% (w/w) to about 0.00010% (w/w) of methionine sulfoxide reductase, one or more of: (i) an inorganic salt; (ii) an amino acid; and (iii) a vitamin, and a pharmaceutically acceptable topical carrier.

In exemplary embodiments, the keratinocyte growth factor is present at an amount of about 0.00003% (w/w) to about 0.00008% (w/w), including at an amount of about 0.00004% (w/w). Suitably, the methionine sulfoxide reductase is present at an amount of about 0.00003% (w/w) to about 0.00008% (w/w), including at an amount of about 0.00004% (w/w).

Exemplary inorganic salts include, but are not limited to, ammonium vanadate, ammonium molybdate, calcium chloride, copper sulfate, ferric nitrate, magnesium chloride, manganese sulfate, potassium chloride, sodium acetate, sodium chloride, sodium metasilicate, sodium phosphate, sodium pyruvate, disodium selenite, stannous chloride, zinc sulfate, and a combination thereof.

Exemplary amino acids include, but are not limited to, alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and a derivative or a combination thereof.

Exemplary vitamins include, but are not limited to, biotin, choline chloride, cyanocobalamin, folic acid, inositol, niacinamide, calcium pantothenate, pyridoxine HCl, riboflavin, thiamine HCl and a combination thereof.

In further embodiments, the formulations further comprise one or more of the following: caffeine, glucose, polyamine, thioctic acid, carnitine, apigenin, stigmasterol and proanthocynadins. In embodiments, the caffeine is present at about 0.001% to about 0.005%, the glucose is present at about 3% to about 8%, the polyamine is present at about 0.00005% to about 0.0003%, the thioctic acid is present at about 0.05% to about 0.2%, the carnitine is present at about 1% to about 3%, wherein the apigenin is present at about 0.05% to about 0.3%, the stigmasterol is present at about 0.01% to about 0.04%, and the proanthocyanidins are present at about 1% to about 5%.

In additional embodiments, the formulation can consisting essentially of about 0.00002% (w/w) to about 0.00010% (w/w) of keratinocyte growth factor, about 0.00002% (w/w) to about 0.00010% (w/w) of methionine sulfoxide reductase, one or more inorganic salts selected from the group consisting of: ammonium vanadate, ammonium molybdate, calcium chloride, copper sulfate, ferric nitrate, magnesium chloride, manganese sulfate, potassium chloride, sodium acetate, sodium chloride, sodium metasilicate, sodium phosphate, sodium pyruvate, disodium selenite, stannous chloride, zinc sulfate and combinations thereof, one or more amino acids selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and derivatives and combinations thereof, one or more vitamins selected from the group consisting of biotin, choline chloride, cyanocobalamin, folic acid, inositol, niacinamide, calcium pantothenate, pyridoxine HCl, riboflavin, thiamine HCl and combinations thereof, caffeine, glucose, polyamine, thioctic acid, carnitine, apigenin, stigmasterol and proanthocynadins, and a pharmaceutically acceptable topical carrier.

In still further embodiments, a topical formulation for promoting hair growth is provided that includes, about 0.00003% (w/w) to about 0.00008% (w/w) of keratinocyte growth factor, about 0.00003% (w/w) to about 0.00008% (w/w) of methionine sulfoxide reductase, caffeine at about 0.001% to about 0.005%, glucose at about 3% to about 8%, polyamine at about 0.00005% to about 0.0003%, thioctic acid at about 0.05% to about 0.2%, carnitine at about 1% to about 3%, apigenin at about 0.05% to about 0.3%, stigmasterol at about 0.01% to about 0.04%, proanthocyanidins present at about 1% to about 5% and a pharmaceutically acceptable topical carrier.

Also provided is a method of increasing one or more characteristics of hair growth, comprising providing a topical formulation for promoting hair growth, comprising about 0.00002% (w/w) to about 0.00010% (w/w) of keratinocyte growth factor, about 0.00002% (w/w) to about 0.00010% (w/w) of methionine sulfoxide reductase, one or more of: (i) an inorganic salt; (ii) an amino acid; and (iii) a vitamin, and a pharmaceutically acceptable topical carrier, applying the topical formulation to a skin surface where the increase in the one or more characteristics of hair growth is desired, and maintaining the topical formulation on the skin surface for a period of at least 1 hour.

Suitably, the applying occurs twice per day for a period of at least 6 weeks.

In embodiments, the increase in one or more characteristics of hair growth is an increase in the length of a hair, the increase in one or more characteristics of hair growth is an increase in the thickness of a hair, the increase in one or more characteristics of hair growth is an increase in the weight of a hair, or the increase in one or more characteristics of hair growth is an increase in the number of hairs per area of the skin surface.

### BRIEF DESCRIPTION OF DRAWINGS

The foregoing and other features and aspects of the present technology can be better understood from the following description of embodiments and as illustrated in the accompanying drawings. The accompanying drawings, which are incorporated herein and form a part of the specification, further serve to illustrate the principles of the present technology. The components in the drawings are not necessarily to scale.

FIGS. 1A-1F show comparative results of untreated scalp and scalp treated with a topical formulation disclosed herein.

FIG. 2 shows comparative results of untreated scalp hair and scalp hair treated with a topical formulation disclosed herein.

FIGS. 3A-3F show results of eyebrows treated with a topical formulation disclosed herein.

FIGS. 4A-4F show results of eyelashes treated with a topical formulation disclosed herein.

FIGS. 5A-5C show results of eyelashes treated with a topical formulation disclosed herein.

FIGS. 6A-6F show comparative results of untreated facial hair (A-C) and facial hair treated with a topical formulation disclosed herein (D-F).

FIGS. 7A-7C show a subject without treatment and after treatment with a topical formulation disclosed herein.

### DETAILED DESCRIPTION OF THE INVENTION

It should be appreciated that the particular implementations shown and described herein are examples and are not intended to otherwise limit the scope of the application in any way.

The published patents, patent applications, websites, company names, and scientific literature referred to herein are hereby incorporated by reference in their entirety to the same extent as if each was specifically and individually indicated to be incorporated by reference. Any conflict between any reference cited herein and the specific teachings of this specification shall be resolved in favor of the latter. Likewise, any conflict between an art-understood definition of a word or phrase and a definition of the word or phrase as specifically taught in this specification shall be resolved in favor of the latter.

As used in this specification, the singular forms “a,” “an” and “the” specifically also encompass the plural forms of the terms to which they refer, unless the content clearly dictates otherwise. The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Technical and scientific terms used herein have the meaning commonly understood by one of skill in the art to which the present application pertains, unless otherwise defined. Reference is made herein to various methodologies and materials known to those of skill in the art.

In embodiments, provided herein are topical formulations for promoting hair growth.

As used herein “topical formulation” refers to any form of a composition or formulation that can be applied to the surface of the skin, including the scalp, face, eyebrows, eyelashes, back, neck, trunk, legs, etc. Topical formulations include liquids, foams, powders, emulsions, sprays, creams, lotions, serums, salves, balms, etc. As described herein, the topical formulations described throughout have been found to promote hair growth on various areas of the body (including scalp, face, eyebrows and eyelashes), in both male and female patients. “Topical formulations” and “formulations” are used interchangeably herein.

In exemplary embodiments, the topical formulations comprise:

about 0.00002% (w/w) to about 0.00010% (w/w) of keratinocyte growth factor;

about 0.00002% (w/w) to about 0.00010% (w/w) of methionine sulfoxide reductase;

one or more of: (i) an inorganic salt; (ii) an amino acid; and (iii) a vitamin; and

a pharmaceutically acceptable topical carrier.

In embodiments, the topical formulations include keratinocyte growth factor (KGF), also known as KGF-1, FGF-7, and heparin binding growth factor. Keratinocyte growth factor is a 167 amino acid residue polypeptide. Keratinocyte growth factor is required for cell division and synthesis of cell components in the hair follicle. KGF is a signal chemical and affects many cellular systems that have KGF receptors on the cell surface, resulting in renewed hair growth accompanied by healthier skin. It also protects from ultraviolet radiation, chemical insult and oxidative stress. KGF used herein is suitably a bioidentical ingredient, produced through bacterial genetic modification and purification.

In exemplary embodiments, the topical formulations include keratinocyte growth factor at an amount of about 0.00002% (w/w) to about 0.00010% (w/w) (as used herein, w/w stands for weight per weight, i.e., weight of the specified ingredient per total weight of the formulation, expressed as a percentage). For example, the topical formulations include keratinocyte growth factor at an amount of about 0.00003% (w/w) to about 0.00009% (w/w), about 0.00003% (w/w) to about 0.00008% (w/w), about 0.00003% (w/w) to about 0.00007% (w/w), about 0.00003% (w/w) to about 0.00006% (w/w), about 0.00003% (w/w) to about 0.00005% (w/w), or about 0.00003% (w), about 0.00004% (w), about 0.00005% (w), about 0.00006% (w), about 0.00007% (w), or about 0.00008% (w). Amounts higher than 0.00010% (w/w) can also be used, including 0.00015%, 0.00020%, 0.00025% and 0.00030%.

Suitably, the formulations also include methionine sulfoxide reductase (MSR), also known as methionine sulfoxide reductase AB (MSR-AB), oxido reductase and sh-polypeptide-107. MRS is an enzyme (255 amino acids, about 26,000 molecular weight) that carries out the enzymatic reduction of methionine sulfoxide, back to methionine, using thioredoxin to catalyze the enzymatic reduction and repair oxidized methionine resides. MSR is suitably a bioidentical ingredient, produced through bacterial genetic modification and purification.

In exemplary embodiments, the topical formulations include methionine sulfoxide reductase at an amount of about 0.00002% (w/w) to about 0.00010% (w/w). For example, the topical formulations include methionine sulfoxide reductase at an amount of about 0.00003% (w/w) to about 0.00009% (w/w), about 0.00003% (w/w) to about 0.00008% (w/w), about 0.00003% (w/w) to about 0.00007% (w/w), about 0.00003% (w/w) to about 0.00006% (w/w), about 0.00003% (w/w) to about 0.00005% (w/w), or about 0.00003% (w), about 0.00004% (w), about 0.00005% (w), about 0.00006% (w), about 0.00007% (w), or about 0.00008% (w). Amounts higher than 0.00010% (w/w) can also be used, including 0.00015%, 0.00020%, 0.00025% and 0.00030%.

In exemplary embodiments, the formulations can contain one or more inorganic salts, including ammonium vanadate, ammonium molybdate, calcium chloride, copper sulfate, ferric nitrate, magnesium chloride, manganese sulfate, potassium chloride, sodium acetate, sodium chloride, sodium metasilicate, sodium phosphate, sodium pyruvate, disodium selenite, stannous chloride, zinc sulfate, and a combination thereof. Various combinations of inorganic salts for use in the formulations can be readily envisioned by a person of ordinary skill in the art.

In additional embodiments, the formulations can contain one or more amino acids, including alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, and a derivative or a combination thereof. Derivatives of such amino acids, as well as combinations in the form of di- or tri-amino acids, are well known to those of ordinary skill in the art.

In additional embodiments, the formulations can contain one or more vitamins, including biotin, choline chloride, cyanocobalamin, folic acid, inositol, niacinamide, calcium pantothenate, pyridoxine HCl, riboflavin, thiamine HCl and a combination thereof. Combinations of such vitamins are well known and can be readily envisioned by those of ordinary skill in the art.

In embodiments, the formulations described herein suitably include one or more of the following additional ingredients: caffeine, glucose, polyamine, thioctic acid, carnitine, apigenin, stigmasterol and proanthocynadins.

In exemplary embodiments, the caffeine is present in the formulations at about 0.001% to about 0.005%, more suitably at about 0.002% to about 0.005%, or about 0.001%, about 0.002%, about 0.003%, about 0.004%, or about 0.005%. Higher amounts of caffeine, including 0.005%-0.010% can also be included in the formulations. Caffeine is suitably included in the topical formulations as a stimulator of hair growth.

Glucose for use in the topical formulations is suitably present at about 3% to about 8%, more suitably at about 4% to about 7%, about 4% to about 6%, or about 3%, about 4%, about 5%, about 6%, about 7% or about 8%. Higher amounts of glucose, including 8%-15%, can also be included in the formulations. Glucose is suitably included in the topical formulations to provide the proper tonicity.

Polyamine is suitably present in the topical formulations at an amount of about 0.00005% to about 0.0003%, more suitably at about 0.00006% to about 0.0002%, about 0.00007% to about 0.0002%, about 0.00008% to about 0.0002%, about 0.00009% to about 0.0002%, or about 0.00007%, about 0.00008%, about 0.00009%, about 0.0001%, about 0.0002% or about 0.0003%. Higher amounts of polyamine, including 0.0003% to about 0.001%, can also be included. Polyamines (e.g., spermidine, putrescine and spermine) are cationic amines that play a role in cellular proliferation and regeneration of tissues.

The topical formulations suitably include thioctic acid (also known as lipoic acid) at about 0.05% to about 0.2%, including at about 0.07% to about 0.2%, about 0.08% to about 0.2%, about 0.09% to about 0.2%, or about 0.07%, about 0.08%, about 0.09%, about 0.10%, about 0.11%, about 0.12%, about 0.13% or about 0.14%. Higher amounts of thioctic acid, including about 0.2%-0.5%, can also be included in the formulations. Thioctic acid is suitably included in the topical formulations to function as an antioxidant. Antioxidants, such as thioctic acid and other antioxidants disclosed herein, disarm reactive oxygen species and complement the activity of chemical antioxidants like vitamin E.

Suitably, carnitine is present in the topical formulations at 0.5% to about 5%, or about 1% to about 4%, about 1% to about 3%, about 1.5% to about 2.5%, or about 1%, about 2%, about 3%, or about 4%. Higher amounts of thioctic acid, including about 5%-8%, can also be included in the topical formulations. Carnitine, an amino acid, is suitably included in the topical formulations to act as a moisturizer, and also aids in the growth of hair. Exemplary forms of carnitine include L-carnitine, which can be supplied in a number of chemical derivatives or salts, such as acetyl carnitine, carnitine tartrate, palmitoyl carnitine, or similar.

Apigenin is suitably present in the topical formulations at about 0.05% to about 0.3%, more suitably about 0.07% to about 0.2%, or about 0.08% to about 0.2%, about 0.09% to about 0.2%, or about 0.07%, about 0.08%, about 0.09%, about 0.1%, about 0.2%, about 0.3% or about 0.4%. Higher amounts of apigenin can also be utilized, including 0.3%-0.8%. Apigenin, a plant-based flavone, suitably increases skin permeability when utilized in the topical formulations disclosed herein.

In embodiments, the topical formulations include stigmasterol at about 0.01% to about 0.04%, including about 0.01% to about 0.03%, about 0.02% to about 0.03%, or about 0.01%, about 0.015%, about 0.02%, about 0.025%, about 0.03%, about 0.035%, or about 0.04%. Stigmasterol, a plant-based sterol, is suitably included in the topical formulations as an anti-inflammatory.

The proanthocyanidins (the main flavonoids found in grape seed extract) are present in the topical formulations at about 1% to about 5%, including at about 2% to about 4%, about 2% to about 3%, or about 1%, about 2%, about 3%, about 4% or about 5%. Higher amounts of proanthocyanidins, including 5%-10%, can also be utilized. Proanthocyanidins, are plant-based phenols, suitably included in the topical formulations as an antioxidant.

As described herein, the topical formulations suitably include a pharmaceutically acceptable topical carrier. In embodiments, a pharmaceutically acceptable topical carrier can include, for example, water, various gums (including guar gum, xanthan gum), glycerin, etc.

Pharmaceutically acceptable topical carriers for use herein suitably include components that when mixed together result in a topical carrier, including various foams, powders, lotions, creams, emulsions, sprays, serums, salves, liquids, etc. “Pharmaceutically acceptable topical carrier” as used herein is any substantially non-toxic carrier, conventionally useable for topical administration of pharmaceuticals in which the various ingredients described herein remain stable and bioavailable when applied directly to skin, mucosal surfaces, or hair follicles. Examples of components useful in a pharmaceutically acceptable topical carrier include moisturizers, humectants, odor modifiers, buffers, pigments, preservatives, Vitamins such as A, C and E, emulsifiers, dispersing agents, wetting agents, odor-modifying agents, gelling agents, stabilizers, propellants, antimicrobial agents, sunscreens, enzymes and the like. Suitable non-limiting examples of additional ingredients include superoxide dismutase, stearyl alcohol, isopropyl myristate, sorbitan monooleate, polyoxyethylene stearate, propylene glycol, water, alkali or alkaline earth lauryl sulfate, methylparaben, oxtyl dimethyl-p-amino benzoic acid (Padimate O), uric acid, reticulin, polymucosaccharides, hyaluronic acids, aloe vera, lecithin, polyoxyethylene sorbitan monooleate, polyethylene glycol, vitamin A or C, tocopherol (vitamin E), alpha-hydroxy of alpha-keto acids such as pyruvic, lactic and glycolic acids, etc.

In further embodiments, provided herein is a topical formulation for promoting hair growth, consisting essentially of about 0.00002% (w/w) to about 0.00010% (w/w) of keratinocyte growth factor, about 0.00002% (w/w) to about 0.00010% (w/w) of methionine sulfoxide reductase, one or more inorganic salts selected from the group consisting of: ammonium vanadate, ammonium molybdate, calcium chloride, copper sulfate, ferric nitrate, magnesium chloride, manganese sulfate, potassium chloride, sodium acetate, sodium chloride, sodium metasilicate, sodium phosphate, sodium pyruvate, disodium selenite, stannous chloride, zinc sulfate and combinations thereof; one or more amino acids selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and derivatives and combinations thereof; one or more vitamins selected from the group consisting of biotin, choline chloride, cyanocobalamin, folic acid, inositol, niacinamide, calcium pantothenate, pyridoxine HCl, riboflavin, thiamine HCl and combinations thereof; caffeine, glucose, polyamine, thioctic acid, carnitine, apigenin, stigmasterol and proanthocynadins; and a pharmaceutically acceptable topical carrier.

In topical formulations that “consist essentially of the recited ingredients, such topical formulations contain the recited components and those that do not materially affect the basic and novel characteristics of the claimed formulations. Components that do not materially affect the basic and novel characteristics of the claimed topical formulations are those that do not limit the ability of the topical formulations to increase hair growth following topical application. Examples of components that would not materially affect the basic and novel characteristics of the claimed topical formulations, and thus could be included in formulations that consist essentially of the recited components, include various moisturizers, humectants, odor modifiers, buffers, pigments, preservatives, Vitamins such as A, C and E, emulsifiers, dispersing agents, wetting agents, odor-modifying agents, gelling agents, stabilizers, propellants, antimicrobial agents, sunscreens, enzymes and the like.

As described herein, in embodiments of the topical formulations that consist essentially of the recited ingredients, the keratinocyte growth factor is suitably present at an amount of about 0.00003% (w/w) to about 0.00008% (w/w), or more suitably about 0.00004% (w/w) (i.e. 0.4 parts per million (ppm)). In such embodiments, the methionine sulfoxide reductase is suitably present at an amount of about 0.00003% (w/w) to about 0.00008% (w/w), including at an amount of about 0.00004% (w/w) (i.e., 0.4 ppm). Additional amounts and ranges of keratinocyte growth factor and methionine sulfoxide reductase that can be included in the topical formulations are described herein.

In exemplary embodiments of the topical formulations that consist essentially of the recited components, the caffeine is present at about 0.001% to about 0.005%, the glucose is present at about 3% to about 8%, the polyamine is present at about 0.00005% to about 0.0003%, the thioctic acid is present at about 0.05% to about 0.2%, the carnitine is present at about 1% to about 3%, the apigenin is present at about 0.05% to about 0.3%, the stigmasterol is present at about 0.01% to about 0.04% and the proanthocyanidins are present at about 1% to about 5%. Additional amounts of these components that can be included in the topical formulations are described herein.

In further embodiments, topical formulations for promoting hair growth are provided herein, suitably comprising about 0.00003% (w/w) to about 0.00008% (w/w) of keratinocyte growth factor, about 0.00003% (w/w) to about 0.00008% (w/w) of methionine sulfoxide reductase, caffeine at about 0.001% to about 0.005%, glucose at about 3% to about 8%, polyamine at about 0.00005% to about 0.0003%, thioctic acid at about 0.05% to about 0.2%, carnitine at about 1% to about 3%, apigenin at about 0.05% to about 0.3%, stigmasterol at about 0.01% to about 0.04%, proanthocyanidins present at about 1% to about 5%, and a pharmaceutically acceptable topical carrier.

As described herein, in exemplary embodiments, the keratinocyte growth factor is present at an amount of about 0.00004% (w/w), and the methionine sulfoxide reductase is present at an amount of about 0.00004% (w/w). Suitably, the topical formulations include amounts of the other components such as caffeine present at about 0.003%, glucose present at about 5%, polyamine present at about 0.0001%, thioctic acid present at about 0.1%, carnitine present at about 2%, apigenin present at about 0.1%, stigmasterol present at about 0.025% and proanthocyanidins present at about 3%.

In additional embodiments, the topical formulations described herein can further include one or more essential fatty acids, as well as other beneficial lipids. Examples of such lipids (and components including such lipids) include, but are not limited, Squalane, Linum Usitatissmum (Flax) Seed Oil, Rosa Canina (Rosehip) Seed Oil, Lecithin, Salvia Hispanica (Chia) Seed Oil, Punica Granatum (Pomegranate) Seed Oil, Argania Spinosa (Argan) Kernel Oil, Tocotrienols, Tocopherols, Astaxanthin, Lycopene, Xanthophyll, R-Alpha Lipoic Acid, Beta-Carotene, Docosahexanoic Acid, Ceramide-3, Cholesteryl Oleyl Carbonate (and) Cholesteryl Nonanoate (and) Cholesteryl Chloride (and) BHT, Phytosterols, Oryzanol.

Suitable components for use in the topical formulations also include the following: Seakelp (Lactobacillus/Kelp Ferment Filtrate) Bioferment, Water, Serenoa Serrulata (Saw Palmetto) Fruit Extract, Soy Isoflavones, Caffeine, Aesculus Hippocastanum (Horse Chestnut) Seed Extract, Carnitine, Niacinamide, Cocos Nucifera (Coconut) Fruit Juice, Urtica Dioica (Nettle) Leaf/Root Extract, Boswellia Serrata Extract, Vitis Vinifera (Grape) Seed Extract, Astragalus, Apigenin, Glutamine, Arginine, Leucine, Serine, Cysteine, Valine, Proline, Lysine, Glycine, Asparagine, Glutamic Acid, Threonine, Alanine, Phenylalanine, Methionine, Aspartic Acid, Tyrosine, Tryptophan, Glucose, sh-Polypeptide-3, Superoxide Dismutase, Catalase, Propylene Glycol (and) Diazolidinyl Urea (and) Methylparaben (and) Propylparaben, Propanediol, Glycerin, PEG-40 Hydrogenated Castor Oil, Biotin, Choline Chloride, Cyanocobalamin, Folic Acid, Inositol, Calcium Pantothenate, Pyridoxine HCl, Riboflavin, Thiamine HCl Ammonium Vanadate, Ammonium Molybdate, Calcium Chloride, Copper Sulfate, Ferric Nitrate, Magnesium Chloride, Manganese Sulfate, Potassium Chloride, Sodium Acetate, Sodium Chloride, Sodium Metasilicate, Sodium Phosphate, Sodium Pyruvate, Disodium Selenite, Stannous Chloride, Zinc Sulfate, Histidine, Isoleucine, Adenine, Thymidine, Hydroxyethylpiperazine Ethane Sulfonic Acid, Thioctic Acid, Spermidine, Xanthan Gum, Cyamopsis Tetragonoloba (Guar) Gum, Polysorbate 20, Phenoxyethanol (and) Chlorphenesin (and) Caprylyl Glycol.

The topical formulations described herein suitably utilize many components, including amino acids and peptides, etc., already present in the human body, thus reducing the potential for side effects or adverse or allergic reactions. The topical formulations described herein are capable of providing the components necessary to promote hair growth directly to a hair follicle, along with components that prevent or reduce oxidative stress, also helping to reduce graying of the hair.

The topical formulations described herein are suitably formulated in a pharmaceutically acceptable topical carrier using well-known formulation methods. For example, the desired topical carrier components are mixed together, including a desired amount of water, suitably at an elevated temperature. During cool down (suitably less than or equal to about 40° C.), keratinocyte growth factor and methionine sulfoxide reductase are suitably added with gentle mixing, resulting in the desired topical formulation. The topical formulations described herein can be stored a room temperature for a period of about 2 years, or longer, particularly if stored under refrigeration.

As described herein, the topical formulations have been found to increase one or more characteristics of hair growth in multiple different patient populations. This demonstrates the broad applicability of the disclosed topical formulations for promoting hair growth, regardless of the underlying reason for hair loss or lack of hair growth.

Thus, in further embodiments, provided herein are methods of increasing one or more characteristics of hair growth, comprising providing a topical formulation for promoting hair growth from those described herein, applying the topical formulation to a skin surface where the increase in the one or more characteristics of hair growth is desired, and maintaining the topical formulation on the skin surface for a period of at least 1 hour.

As described throughout, suitably the topical formulations described herein include about 0.00002% (w/w) to about 0.00010% (w/w) of keratinocyte growth factor, about 0.00002% (w/w) to about 0.00010% (w/w) of methionine sulfoxide reductase, one or more of: (i) an inorganic salt; (ii) an amino acid; and (iii) a vitamin and a pharmaceutically acceptable topical carrier.

As used herein a “characteristic of hair growth” includes one or more of the following: an increase in length of a hair, an increase in thickness of a hair, an increase in weight of a hair, or an increase in the number of hairs per area of a skin surface. The methods that increase the “characteristics of hair growth” suitably increase the characteristic by at least about 10%, when compared to the characteristic of hair growth for a patient/hair that was treated with a control (i.e., a topical formulation including only pharmaceutically acceptable topical carrier(s), or completely untreated). Suitably, the increase in the characteristic for the treated hair increases by at least about 15%, at least about 20%, at least about 25%, at least about 30%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 200%, at least about 250%, at least about 300%, etc., relative to a control.

The methods of treatment described herein suitably include applying the topical formulation to a skin surface where the increase in the one or more characteristics of hair growth is desired. That is the skin surface of a mammalian (including human) face, scalp, eyebrows, eyelashes, arm, trunk, back, legs, etc. The skin surface where the topical formulations are applied suitably includes hair follicles into which the topical formulations penetrate to provide the desired increase of a hair characteristic. Suitably, the applying occurs twice per day for a period of at least 6 weeks, or in other embodiments, 2-4 times per day, for 4 weeks, 6 weeks, 8 weeks, 10 weeks or 12 weeks. Longer periods of treatment (i.e., greater than 12 weeks) can also be utilized.

As described herein, the topical formulations are suitably maintained on the skin surface for a period of at least 1 hour. That is, the topical formulations are suitably not washed off, rubbed off, or otherwise removed from the skin surface. If undesired removal does occur, the topical formulations can simply be re-applied. In exemplary embodiments, the topical formulations are maintained on the skin surface for a period of at least 4 hours, at least 6 hours, at least 8 hours, including overnight.

In additional embodiments, the topical applications can be applied to a skin surface to reduce or prevent the appearance of gray hair. Suitably, the topical formulations are applied to a skin surface, including a hair follicle, that produces or is capable of producing a gray hair. The topical formulations are able to penetrate the hair follicle and reduce the gray color of a produced hair or completely eliminate the production of a gray hair. Frequency of treatment, times for maintaining treatment, and overall treatment durations are similar to those described herein for treatment of characteristic of hair growth. The topical formulations described herein are believed to help repair protein damage caused by reactive oxygen species that escaped the first line of antioxidant defense. The topical formulations are believed to repair oxidized amino acids, including methionine, in a damaged tyrosinease protein so that the enzyme can continue to catalyze the first key step in the synthesis of melanin, thereby reducing/preventing/treating, gray hair.

The topical formulations described herein can be applied to a skin surface using various devices or applicators, depending on the form of the formulation (i.e., spray, foam, serum, lotion, cream, etc.). For example, the topical formulations can be applied using a sponge, a towel, a spray bottle, a foaming applicator, or other suitable applicators, including simply using the hands/fingers. Depending on the location of the desired treatment, the size of the applicator can be tailored to provide maximal coverage. For example, a small applicator can be used to apply the topical formulations to the eyebrows, making sure it reaches the skin. A similar applicator can be used for eyelashes, where the product is applied to the edge of the eyelid. For application to the face, i.e., beard area, the topical formulations can be applied with a spray or similar applicator and massaged on the areas lacking or having inadequate hair growth. Similar applicators and massage can be used on the scalp, again in areas requiring attention.

The methods of treatment and topical formulations described herein, in addition for use in patients that are generally healthy, can also be used on cancer patients who are subject to cellular injury, i.e., from radiation or chemotherapy to treat the cancer. The methods herein can allow such patients to retain some hair, or eyebrows or eyelashes, the loss or which can be upsetting. The topical formulations and methods can also be utilized on various mammals, including domesticated pets, such as cats and dogs, as well as in a laboratory setting (i.e., rat, mouse, primate), wherein increased hair growth may be desired in order to test the effect of a new compound, treatment, device, etc.

### EXAMPLES

### Example 1

### Treatment of Scalp Hair

A topical formulation having the following composition was prepared:

| Product group | Part | Notes | UoM | Quantity |
|---|---|---|---|---|
| #AG-00004 Liquid - Misc | #A-01519 Distilled Water | | gm | 3098.4 |
| #AG-00009 Powder & | #A-01848 Guar Gum | | gm | 15.2 |
| Dry Misc | | | | |
| #AG-00009 Powder & | #A-03257 Xanthan Gum | | gm | 9.2 |
| Dry Misc | | | | |
| #AG-00004 Liquid - Misc | #A-01816 Glycerin (Natural) | | gm | 80 |
| #AG-00009 Powder & | #A-00281 Caffeine | | gm | 40 |
| Dry Misc | (Anhydrous) | | | |
| #AG-00009 Powder & | #A-01543 EDTA | Disodium Salt | gm | 2 |
| Dry Misc | | Dihydrate | | |
| #AG-00009 Powder & | #A-03942 Keratinocyte | | gm | 36 |
| Dry Misc | Medium SA Custom | | | |
| #AG-00009 Powder & | #A-00347 Carnitine (L) | | gm | 2 |
| Dry Misc | | | | |
| #AG-00009 Powder & | #A-00416 Coconut Endosperm | | gm | 4 |
| Dry Misc | (Cococin) | | | |
| #AG-00009 Powder & | #A-02583 Niacinamide - | | gm | 4 |
| Dry Misc | Vitamin B3 | | | |
| #AG-00009 Powder & | #A-02576 Nettle Root | | gm | 2 |
| Dry Misc | | | | |
| #AG-00009 Powder & | #A-00197 Boswellia Serrata | | gm | 12 |
| Dry Misc | | | | |
| #AG-00009 Powder & | #A-01778 Fucoidan (85%) | | gm | 12 |
| Dry Misc | | | | |
| #AG-00006 Product - | #A-02841 SA Ferment | | gal | 0.0785 |
| Components | | | | |
| #AG-00004 Liquid - Misc | #A-00004 1,3-Propanediol - | | gm | 120 |
| | NatureSilk | | | |
| #AG-00009 Powder & | #A-01832 Grape Seed | | gm | 4 |
| Dry Misc | Proanthocyanidins | | | |
| #AG-00052 Ingredient | #A-02671 PEG 40 | | gm | 160 |
| | Hydrogenated Castor Oil | | | |
| #AG-00009 Powder & | #A-02865 Saw Palmetto | | gm | 40 |
| Dry Misc | | | | |
| #AG-00009 Powder & | #A-00420 Coenzyme Q10 | | gm | 4 |
| Dry Misc | | | | |
| #AG-00004 Liquid - Misc | #A-02603 Optiphen Plus | | gm | 37.2 |
| #AG-00013 Product - | #A-00082 SA - Antioxidant | | Kg | 0.004 |
| Serum | Booster Bulk | | | |
| #AG-00020 Hi-Tech | #A-01958 Keratinocyte | | mg | 1.6 |
| | Growth Factor (KGF) | | | |
| #AG-00020 Hi-Tech | #A-03015 Superoxide | | mg | 3.2 |
| | Dismutase (SOD) | | | |
| #AG-00020 Hi-Tech | #A-04570 Methionine | | mg | 1.6 |
| | Sulfoxide Reductase A/B - | | | |
| | (MSR - A/B) | | | |

A trial was designed to measure the effect of the topical formulation on the thickness, density (hairs per area of skin) and length, of a human patient's hair over a 12 week period.

### Methods

Eleven participants, ten female and one male, received a detailed questionnaire about their perception of their hair thickness and density. They were asked to individually record their perceptions on a scale of 1 to 5, with regard to thickness and density of their hair. High definition photographs were then taken at this time to act as a control for the trials. Participants were then asked to use the topical formulation noted above, twice per day for twelve weeks, applying the serum to the scalp. Two participants failed to complete the trial and are not included in the results.

Twelve weeks into the trial, a complete survey was given to participants to gauge progress and new sets of comparison photographs were taken. Detailed observations of high definition photographs were utilized to determine if the thickness of individual hairs had increased, if new hairs were observed, and if average hair length increased in the course of 12 weeks.

### Results and Conclusions

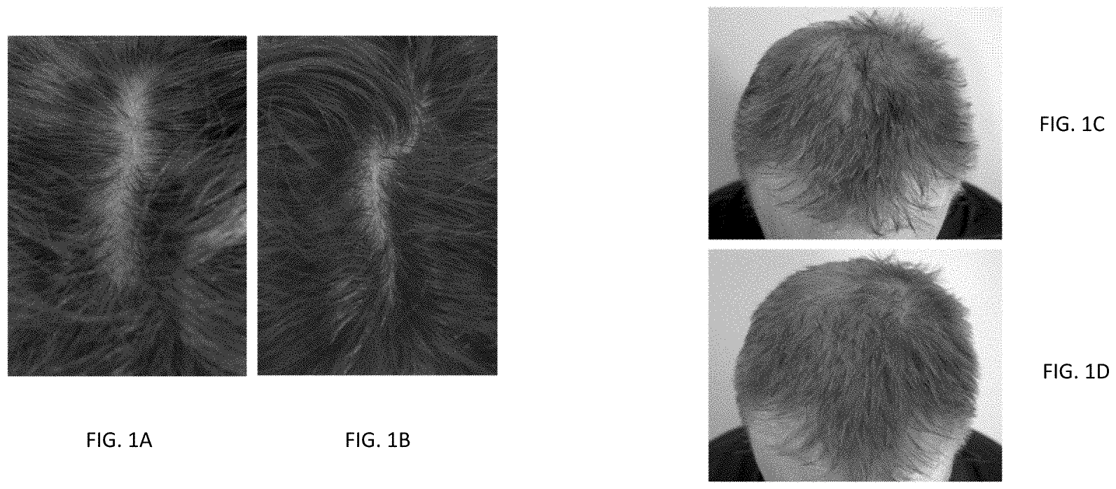

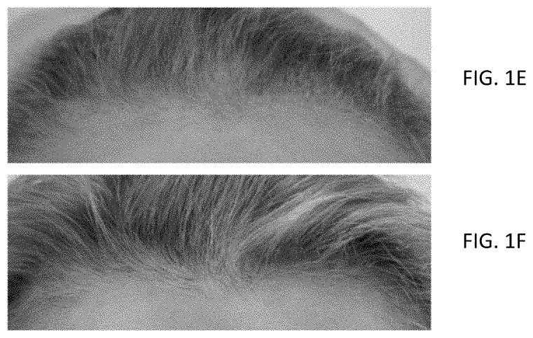

FIGS. 1A, 1C and E, show patients before application of the topical formulation. FIGS. 1B, 1D and 1F, show the same patients, respectively, following the 12 week treatment protocol. In each case, an increase in hair density, hair length, hair thickness and number of hairs can be visually noted.

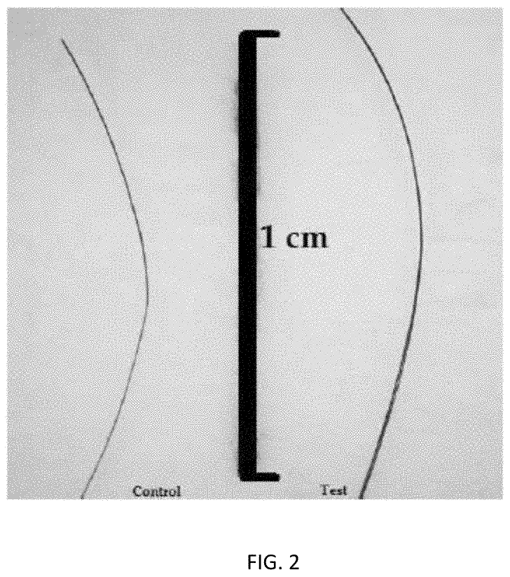

FIG. 2 shows a comparison of a scalp hair prior to treatment on the left (control), and a hair treated with the topical formulation for 12 weeks (right-test). The increase in the thickness of the hair can clearly be seen.

Table 1 below shows the compiled results of the patient satisfaction surveys.

TABLE 1
Patient Survey Results

| # | Age | Sex | Change Observed | Thickness | Quantity | Length |
|---|---|---|---|---|---|---|
| 1 | 67 | F | Not Sure | Not Sure | Not Sure | Not Sure |
| 2 | 66 | F | Yes | Yes | Yes | Yes |
| 3 | 41 | M | Yes | No | Yes | No |
| 4 | 62 | F | Yes | No | No | Yes |
| 5 | 23 | F | Yes | Yes | Yes | Yes |
| 6 | 50 | F | Yes | No | Yes | No |
| 7 | 62 | F | Yes | Yes | No | Yes |
| 8 | 67 | F | No | No | No | No |
| 9 | 57 | F | Yes | Yes | Yes | No |

78% of the participants (7/9) perceived positive changes in one or more categories of characteristics of hair growth. From these results, it can be concluded that the topical formulation improved the texture, thickness and density of the hair on the scalp.

### Example 2

### Treatment of Eyebrow and Eyelash Hair

### Methods

Week 0: Sixteen human participants received a detailed questionnaire about their perception of their brows and lashes, as well as their use of brow or lash enhancers in the past. They were asked to individually record their perceptions on a scale of 1 to 5, with regard to thickness and density of their brows and lashes, respectively (shown in Table 2). High definition photographs were then taken at this time to act as a control for the trials.

Participants were then asked to use the topical formulation of Example 1 twice per day for twelve weeks, applying the formulation to the skin at the brow and the lash line.

Week 2: Two weeks into the trial, brief surveys regarding the brow and lash products were sent to participants to test for speed of participant response to the products. The survey asked if using the formulation made their brows and lashes: feel more nourished, feel stronger, have enhanced appearance, appear thicker (denser), and appear lengthened. Additionally, respondents were asked if they felt there were real results with using the product, as well as if they would recommend it (Table 3).

Week 6: Six weeks into the trial, a complete survey was sent to participants to gauge progress (Table 2) and new sets of comparison photographs were taken. Detailed observations of high definition photographs were utilized to determine if the thickness of individual hairs had increased, if new hairs were observed, and if average lash length increased in the course of 6 weeks.

Week 12: Twelve weeks into the trial, a complete survey was sent to participants to gauge progress (Table 2) and new sets of comparison photographs were taken. Detailed observations of high definition photographs were utilized to determine if the thickness of individual hairs had increased, if new hairs were observed, and if average lash length increased in the course of 12 weeks.

### Results and Conclusions

Table 2 shows the results of the participant survey related to eyebrow treatment:

TABLE 2

| | | | Brow Thickness | | | Brow Density | | | Perceived Real Results | |
|---|---|---|---|---|---|---|---|---|---|---|
| | | | Week | Week | Week | Week | Week | Week | Week | Week |
| ID No. | Age | Gender | 0 | 6 | 12 | 0 | 6 | 12 | 6 | 12 |
| 1 | 24 | F | 3 | 4 | 5 | 3 | 3 | 5 | Y | Y |
| 2 | 28 | F | 4 | 3 | 5 | 3 | 4 | 5 | Y | Y |
| 3 | 36 | F | Did not participate in Brow Trial | | | | | | | |
| 4 | 31 | F | Did not participate in Brow Trial | | | | | | | |
| 5 | 42 | F | 4 | 4 | 5 | 3 | 4 | 4 | Y | Y |
| 6 | 62 | F | Missing Week 0 Scores | | | | | | Y | Y |
| 7 | 59 | F | 2 | 4 | 3 | 2 | 4 | 3 | Y | Y |
| 8 | 32 | F | 3 | 4 | 4 | 3 | 4 | 4 | Y | Y |
| 9 | 63 | F | Unable to contact after initial interview | | | | | | | |
| 10 | 62 | F | 3 | 3 | 4 | 3 | 3 | 3 | Y | Y |
| 11 | 56 | F | 3 | 3 | 3 | 2 | 3 | 3 | N | N |
| 12 | 22 | F | 3 | 3 | 3 | 3 | 3 | 3 | N | Y |
| 13 | 62 | F | 3 | 3 | 3 | 3 | 3 | 3 | Y | Y |
| 14 | 55 | F | 3 | 3 | 3 | 3 | 3 | 3 | Y | Y |
| 15 | 44 | F | 3 | 4 | 3 | 3 | 4 | 3 | N | Y |
| 16 | 49 | F | 3 | 3 | 4 | 3 | 3 | 4 | Y | Y |
| | | | Average Response | | | | | | | |
| | | | 3.1 | 3.4 | 3.8 | 2.8 | 3.4 | 3.6 | 10 of 13 | 12 of 13 |
| | | | | | | | | | positive | positive |

Table 3 shows the results of the participant survey related to eyelash treatment:

TABLE 3

| | | | Lash Thickness | | | Lash Density | | | Perceived Real Results | |
|---|---|---|---|---|---|---|---|---|---|---|
| | | | Week | Week | Week | Week | Week | Week | Week | Week |
| ID No. | Age | Gender | 0 | 6 | 12 | 0 | 6 | 12 | 6 | 12 |
| 1 | 24 | F | 4 | 5 | 4 | 4 | 5 | 4 | Y | Y |
| 2 | 28 | F | 3 | 3 | 4 | 3 | 3 | 4 | Y | Y |
| 3 | 36 | F | 3 | 3 | 3 | 3 | 3 | 3 | Y | Y |
| 4 | 31 | F | 2 | 3 | 4 | 2 | 3 | 4 | Y | Y |
| 5 | 42 | F | 3 | 3 | 4 | 3 | 4 | 4 | Y | Y |
| 6 | 62 | F | 2 | 3 | 3 | 2 | 3 | 3 | Y | Y |
| 7 | 59 | F | 2 | 4 | 3 | 2 | 4 | 3 | Y | Y |
| 8 | 32 | F | 3 | 4 | 4 | 3 | 4 | 4 | Y | Y |
| 9 | 63 | F | Unable to contact after initial interview | | | | | | | |
| 10 | 62 | F | 3 | 3 | 4 | 2 | 3 | 3 | Y | Y |
| 11 | 56 | F | 1 | 2 | 2 | 2 | 2 | 2 | N | N |
| 12 | 22 | F | 3 | 3 | 4 | 4 | 3 | 3 | Y | Y |
| 13 | 62 | F | 2 | 2 | 3 | 2 | 2 | 3 | Y | Y |
| 14 | 55 | F | 3 | 3 | 3 | 3 | 3 | 3 | Y | Y |
| 15 | 44 | F | 3 | 3 | 3 | 3 | 3 | 3 | Y | Y |
| 16 | 49 | F | 2 | 3 | 3 | 3 | 3 | 3 | Y | Y |
| | | | Average Response | | | | | | | |
| | | | 2.6 | 3.1 | 3.4 | 2.7 | 3.2 | 3.3 | 14 of 15 | 14 of 15 |
| | | | | | | | | | positive | positive |

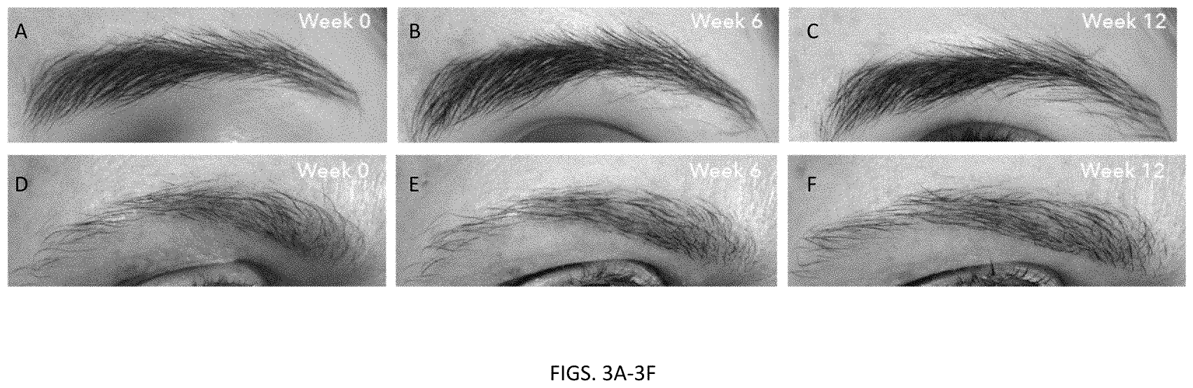

FIGS. 3A-3C and 3D-3F show the results from two patients, over the course of the 12 week period, for the eyebrow treatment. As can be seen, analysis of high definition pictures showed increased thickness of individual hairs. New hair growth and longer hair length were also observed.

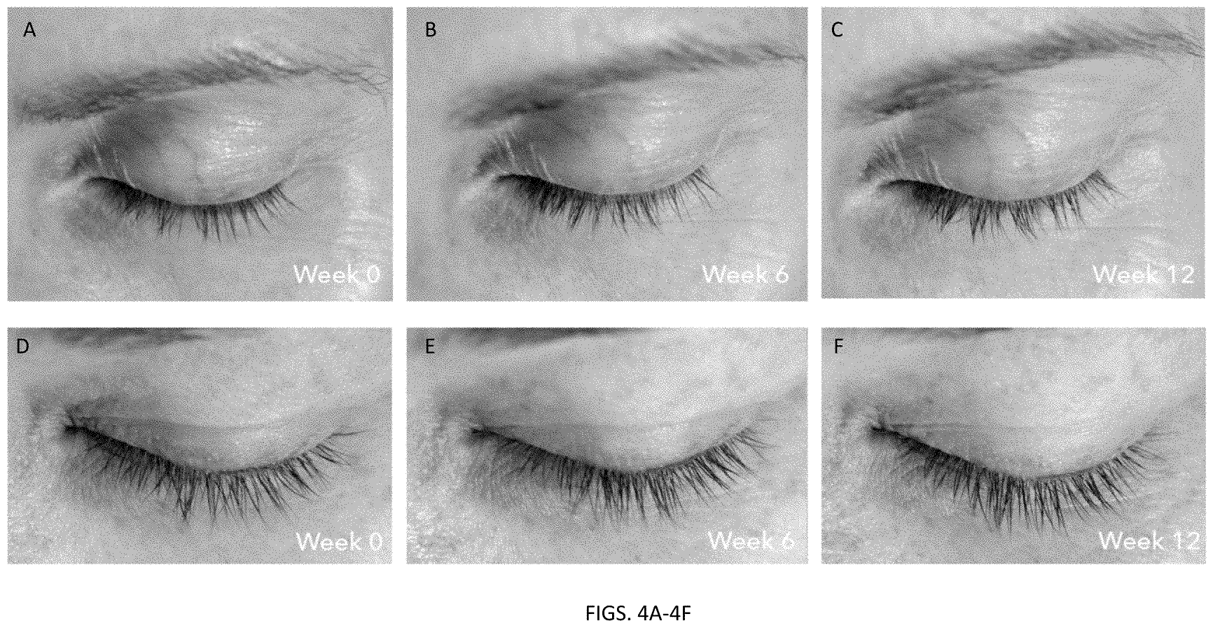

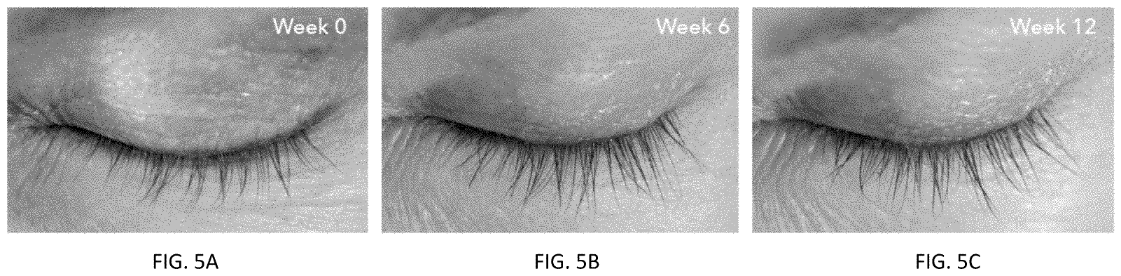

FIGS. 4A-4C, 4D-4F and 5A-5C, show the results from three patients, over the course of the 12 week period, for the eyelash treatment. As can be seen, analysis of high definition pictures showed increased density and thickness over the period of use. New hair growth and longer length hair were also observed.

Week 6, Tables 2 and 3: Of the participants, 14 out of 15 (93%) perceived real, positive results to their lashes in six weeks. 10 out of 13 (77%) perceived real, positive results to their brows in six weeks.

On a scale of 1 to 5 participants' average response in 6 weeks:
Lash Thickness—increase from 2.6 to 3.1 (19% change)
Lash Density—increase from 2.7 to 3.2 (19% change)
Brow Thickness—increase from 3.1 to 3.4 (10% change)
Brow Density—increase from 2.8 to 3.4 (21% change)

Week 12, Tables 2 and 3: Of the participants, 14 out of 15 (93%) perceived real, positive results to their lashes in 12 weeks. 12 out of 13 (92%) perceived real, positive results to their brows in 12 weeks.

On scale of 1 to 5 participants' average response in 12 weeks:
Lash Thickness—increase from 2.6 to 3.4 (31% change)
Lash Density—increase from 2.7 to 3.3 (22% change)
Brow Thickness—increase from 3.1 to 3.8 (23% change)
Brow Density—increase from 2.8 to 3.6 (29% change)

Over 70% of participants noted results by the second week of the trial. After 6 weeks, 14 out of 15 (93%) participants perceived real positive results to their lashes, and 10 out of 13 (77%) had similar feelings about their brows. After 12 weeks, 14 out of 15 (93%) participants perceived real positive results to their lashes, and 12 out of 13 (92%) had similar feelings about their brows.

### Example 3

### Treatment of Beard Hair

### Methods

### First Stage—Control—No Hair Care Serum

Nine human subjects, differing in ethnicities (including Hispanic, Hispanic/Native American, Caucasian, African American), ranging from ages 24 to 44, were asked to razor-shave their facial hair at the beginning of the control trial and to not shave their facial hair for a subsequent period of 24 days. They each had photographs taken of their respective facial hair at the end of the 24 day time period using a Sony STL-A58 digital camera.

Each participant then had their respective facial hair shaved off using Oster Aspire Clippers on a setting of zero and the hair was collected using a Beard King. Photographs of each participant were also taken after shaving. The hair was carefully collected and weighed on a Mettler Toledo ME1002E laboratory scale. Results were recorded and logged (see Table 4).

### Second Stage—Effect of the Topical Formulation

The nine freshly-shaved participants were then asked to not shave for 24 days and to apply the topical formulation of Example 1, twice a day for the next 24 days; once in the morning and once at night—applying the topical formulation directly to the skin (not only to the hair). With the exception of subjects 1 and 4, the remaining 7 subjects were able to finish the study. Each participant then had their respective facial hair shaved off as per the Control stage (using Oster Aspire Clippers on a setting of zero and the hair was collected using a Beard King). Photographs of each participant were taken after shaving occurred. Photographs of each participant were also taken before shaving. The hair was carefully collected and weighed on a Mettler Toledo ME1002E laboratory scale. Results were recorded and logged (see Table 4).

### Results

### Comparison of Treated and Non-Treated Hair

Table 4 below shows the results from the beard growth study:

TABLE 4
Comparison of hair weight for control and treated stages

| | | | | Trial | Stage 1 | Stage 2 | Change in | |
|---|---|---|---|---|---|---|---|---|
| | | | | Duration | Measurement | Measurement | Measurement | % |
| Subject | Ethnicity | Sex | Age | (Days) | (Control) | (Treated) | (Δ) | Change |
| 1 | African | Male | 24 | 27 | 0.04 g | 0.05 g | +0.01 g | +1% |
| | American | | | | | | | |
| 2 | Hisp./Nat. | Male | 24 | 24 | 0.04 g | 0.04 g | +0.00 g | 0% |
| | American | | | | | | | |
| 3 | Hispanic | Male | 26 | 24 | 0.36 g | 0.37 g | +0.01 g | +1% |
| 4 | Caucasian | Male | 30 | 20 | 0.87 g | 0.95 g | +0.08 g | +8% |
| 5 | Caucasian | Male | 32 | 24 | 0.39 g | 0.41 g | +0.02 g | +2% |
| 6 | African | Male | 32 | 24 | 0.57 g | 0.61 g | +0.04 g | +4% |
| | American | | | | | | | |
| 7 | Caucasian | Male | 37 | 24 | 1.01 g | 1.13 g | +0.12 g | +12% |
| 8 | Caucasian | Male | 38 | 24 | 1.36 g | 1.39 g | +0.03 g | +3% |
| 9 | Hispanic | Male | 44 | 24 | 1.66 g | 1.74 g | +0.08 g | +8% |

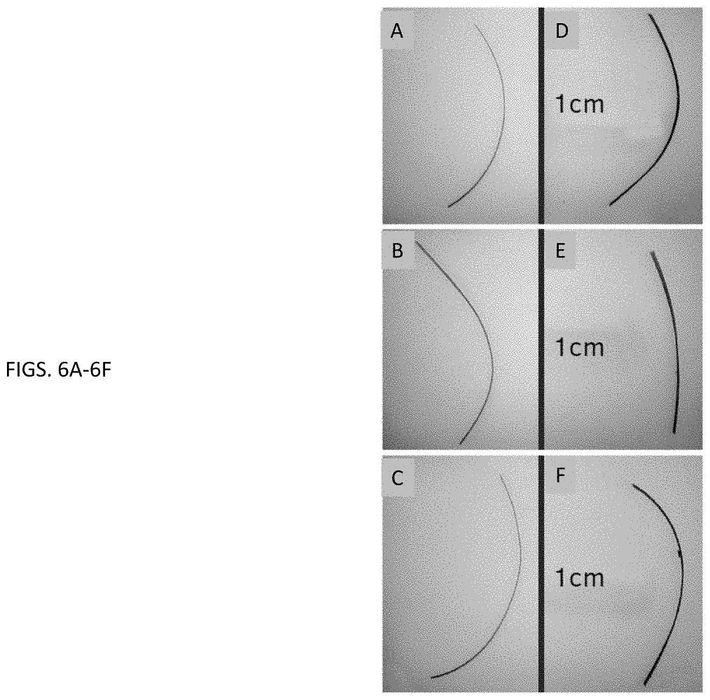

Three sets of facial hair from subject 8 were collected from the control and treated stages. Hair strands from each respective stage were picked at random and compared under the OT-M Opti-Tek Scope at 300× magnification with 1.7× digital zoom. FIGS. 6A-6C shows the hairs from the control stage, FIGS. 6D-6F, show hairs from the treated stage. A one centimeter (1 cm) metric mark is shown for size reference. FIGS. 6A-6F illustrate the increase in the thickness of hair from pre- (i.e., control (FIGS. 6A-6C)) to post-treatment (FIGS. 6D-6F).

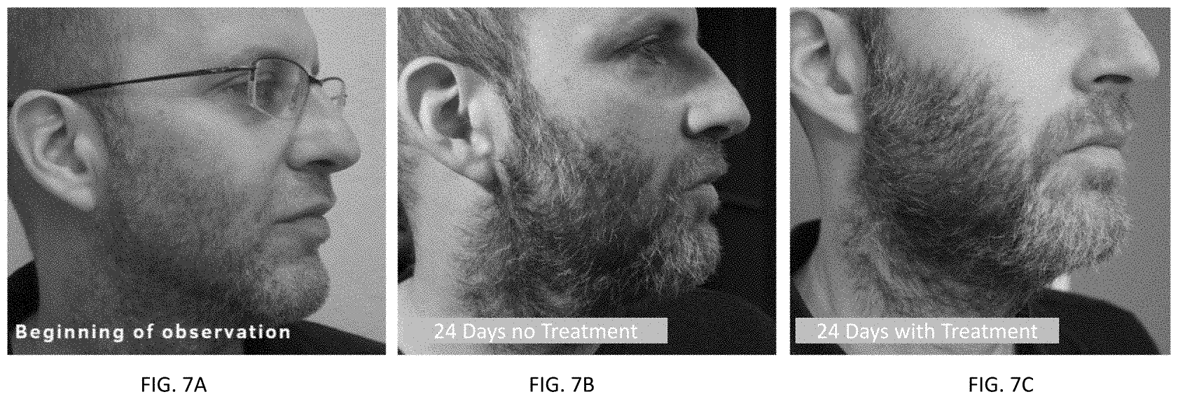

The topical formulations were found to increase the weight of the beard hair in all but one patient. The average hair weight increase was 4.3% overall. In patients over age 30, the average weight increase was 6.1%. FIGS. 7A-7C show the effects of the topical formulation, following 24 days of treatment (FIG. 7A vs. 7C), as compared to the control stage (no topical formulation), shown in FIG. 7A vs. 7B.

### REFERENCES

Jang, J H (2005) Stimulation of human hair growth by the recombinant human keratinocyte growth factor-2 (KGF-2). Biotechnology Letters, 27:749-52.

Guo, Lifei; Degenstein, Linda; Fuchs, Elaine (1996) Keratinocyte growth factor is required for hair development but not for wound healing. Genes & Development 10:165-75.

Karvinen, S; Pasonen-Seppanen, S; Hyttinen, J M T; Pienimaki, J P; Torronen, K, Jokela, T A; Tammi, M I, Tammi, R (2003) Keratinocyte growth factor stimulates migration and hyaluronan synthesis in the epidermis by activation of keratinocyte hyaluronan synthases 2 and 3. J Biol Chem, 278:49495-49504

Danilenko, Dimitry M.; Ring, Brian D.; Yanagihara, Donna; Benson, William; Wiemann, Bernadette; Starnes, Charles O.; Pierce, Glenn F. (1995) Keratinocyte growth factor is an important endogenous mediator of hair follicle growth, development, and differentiation. Normalization of the nu/nu follicular differentiation defect and amelioration of chemotherapy induced alopecia. American Journal of Pathology 147: 145-54.

Braun, S, Krampert, M, E, Bodo, E; Kumin, A Born-Berclaz, C; Paus, R Werner, S (2006) Keratinocyte growth factor protects epidermis and hair follicles from cell death induced by UV irradiation, chemotherapeutic or cytotoxic agents. J. Cell Science, 119: 4841-4849.

Moskovitz, J., Bar-Noy, S., Williams, M. W., Requena, J., Berlett, S. B. & Stadtman, E. R. (2001) Methionine sulfoxide reduc-tase (MsrA) is a regulator of antioxidant defense and lifespan in mammals, Proc. Natl. Acad. Sci. USA 98, 12920-12925.

Wood, J. M., Decker, H., Hartmann, H., Chavan, B., Rokos, H., Spencer, J. D., Hasse, S. Thornton, M. J., Shalbaf, M., Paus, R., Schallreuter, K. U. (2009) Senile hair graying: H202-mediated oxidative stress affects human hair color by blunting methioninesulfoxide repair. FASEB Journal, 23(7):2065-75.

It is to be understood that while certain embodiments have been illustrated and described herein, the claims are not to be limited to the specific forms or arrangement of parts described and shown. In the specification, there have been disclosed illustrative embodiments and, although specific terms are employed, they are used in a generic and descriptive sense only and not for purposes of limitation. Modifications and variations of the embodiments are possible in light of the above teachings. It is therefore to be understood that the embodiments may be practiced otherwise than as specifically described.

While various embodiments have been described above, it should be understood that they have been presented only as illustrations and examples of the present technology, and not by way of limitation. It will be apparent to persons skilled in the relevant art that various changes in form and detail can be made therein without departing from the spirit and scope of the present technology. Thus, the breadth and scope of the present technology should not be limited by any of the above-described embodiments, but should be defined only in accordance with the appended claims and their equivalents. It will also be understood that each feature of each embodiment discussed herein, and of each reference cited herein, can be used in combination with the features of any other embodiment. All patents and publications discussed herein are incorporated by reference herein in their entirety.

What is claimed is:

1. A topical formulation for promoting hair growth, comprising:
a. about 0.00002% (w/w) to about 0.00010% (w/w) of keratinocyte growth factor;
b. about 0.00002%% (w/w) to about 0.00010% (w/w) of methionine sulfoxide reductase;
c. one or more of: (i) an inorganic salt; (ii) an amino acid; and (iii) a vitamin; and
d. a pharmaceutically acceptable topical carrier.

2. The topical formulation of claim 1, wherein the keratinocyte growth factor is present at an amount of about 0.00003% (w/w) to about 0.00008% (w/w).

3. The topical formulation of claim 1, wherein the keratinocyte growth factor is present at an amount of about 0.00004% (w/w).

4. The topical formulation of claim 1, wherein the methionine sulfoxide reductase is present at an amount of about 0.00003% (w/w) to about 0.00008% (w/w).

5. The topical formulation of claim 1, wherein the methionine sulfoxide reductase is present at an amount of about 0.00004% (w/w).

6. The topical formulation of claim 1, wherein the inorganic salt is ammonium vanadate, ammonium molybdate, calcium chloride, copper sulfate, ferric nitrate, magnesium chloride, manganese sulfate, potassium chloride, sodium acetate, sodium chloride, sodium metasilicate, sodium phosphate, sodium pyruvate, disodium selenite, stannous chloride, zinc sulfate, or a combination thereof.

7. The topical formulation of claim 1, wherein the amino acid is alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine, or a derivative or a combination thereof.

8. The topical formulation of claim 1, wherein the vitamin is biotin, choline chloride, cyanocobalamin, folic acid, inositol, niacinamide, calcium pantothenate, pyridoxine HCl, riboflavin, thiamine HCl or a combination thereof.

9. The topical formulation of claim 1, further comprising one or more of the following: caffeine, glucose, polyamine, thioctic acid, carnitine, apigenin, stigmasterol and proanthocynadins.

10. The topical formulation of claim 9, wherein the caffeine is present at about 0.001% to about 0.005%, the glucose is present at about 3% to about 8%, the polyamine is present at about 0.00005% to about 0.0003%, the thioctic acid is present at about 0.05% to about 0.2%, the carnitine is present at about 1% to about 3%, wherein the apigenin is present at about 0.05% to about 0.3%, the stigmasterol is present at about 0.01% to about 0.04%, and the proanthocyanidins are present at about 1% to about 5%.

11. The topical formulation of claim 1, consisting essentially of:
a. about 0.00002% (w/w) to about 0.00010% (w/w) of keratinocyte growth factor;
b. about 0.00002% (w/w) to about 0.00010% (w/w) of methionine sulfoxide reductase;
c. one or more inorganic salts selected from the group consisting of: ammonium vanadate, ammonium molybdate, calcium chloride, copper sulfate, ferric nitrate, magnesium chloride, manganese sulfate, potassium chloride, sodium acetate, sodium chloride, sodium metasilicate, sodium phosphate, sodium pyruvate, disodium selenite, stannous chloride, zinc sulfate and combinations thereof;
d. one or more amino acids selected from the group consisting of: alanine, arginine, asparagine, aspartic acid, cysteine, glutamic acid, glutamine, glycine, histidine, isoleucine, leucine, lysine, methionine, phenylalanine, proline, serine, threonine, tryptophan, tyrosine, valine and derivatives and combinations thereof;
e. one or more vitamins selected from the group consisting of biotin, choline chloride, cyanocobalamin, folic acid, inositol, niacinamide, calcium pantothenate, pyridoxine HCl, riboflavin, thiamine HCl and combinations thereof;
f. caffeine, glucose, polyamine, thioctic acid, carnitine, apigenin, stigmasterol and proanthocynadins; and
g. a pharmaceutically acceptable topical carrier.

12. A topical formulation for promoting hair growth, comprising:
a. about 0.00003% (w/w) to about 0.00008% (w/w) of keratinocyte growth factor;
b. about 0.00003% (w/w) to about 0.00008% (w/w) of methionine sulfoxide reductase;
c. caffeine at about 0.001% to about 0.005%;
d. glucose at about 3% to about 8%;
e. polyamine at about 0.00005% to about 0.0003%;
f. thioctic acid at about 0.05% to about 0.2%;
g. carnitine at about 1% to about 3%;
h. apigenin at about 0.05% to about 0.3%;
i. stigmasterol at about 0.01% to about 0.04%;
j. proanthocyanidins present at about 1% to about 5%; and
k. a pharmaceutically acceptable topical carrier.

13. The topical formulation of claim 12, wherein the keratinocyte growth factor is present at an amount of about 0.00004% (w/w).

14. The topical formulation of claim 12, wherein the methionine sulfoxide reductase is present at an amount of about 0.00004% (w/w).

15. The topical formulation of claim 12, wherein the caffeine is present at about 0.003%, the glucose is present at about 5%, the polyamine is present at about 0.0001%, the thioctic acid is present at about 0.1%, the carnitine is present at about 2%, the apigenin is present at about 0.1%, the stigmasterol is present at about 0.025%, and the proanthocyanidins are present at about 3%.

16. A method of increasing one or more characteristics of hair growth, comprising:
a. providing a topical formulation for promoting hair growth, comprising:
about 0.00002% (w/w) to about 0.00010% (w/w) of keratinocyte growth factor;
about 0.00002% (w/w) to about 0.00010% (w/w) of methionine sulfoxide reductase;
one or more of: (i) an inorganic salt; (ii) an amino acid; and (iii) a vitamin; and
a pharmaceutically acceptable topical carrier;
b. applying the topical formulation to a skin surface where the increase in the one or more characteristics of hair growth is desired; and
c. maintaining the topical formulation on the skin surface for a period of at least 1 hour.

17. The method of claim 16, wherein the applying occurs twice per day for a period of at least 6 weeks.

18. The method of claim 16, wherein the increase in one or more characteristics of hair growth is an increase in the length of a hair, the increase in one or more characteristics of hair growth is an increase in the thickness of a hair, the increase in one or more characteristics of hair growth is an increase in the weight of a hair, or the increase in one or more characteristics of hair growth is an increase in the number of hairs per area of the skin surface.
